(19) United States
(12) Patent Application Publication
(10) Pub. No.: US 2024/0327322 A1
(43) Pub. Date: Oct. 3, 2024

(54) Crosslinked Polymers with Tunable Coefficients of Thermal Expansion

(71) Applicants: National Technology & Engineering Solutions of Sandia, LLC, Albuquerque, NM (US); The Board of Regents of the University of Texas Systems, Austin, TX (US)

(72) Inventors: Erica Marie Redline, Albuquerque, NM (US); Chad Staiger, Albuquerque, NM (US); Jeffrey C. Foster, Albuquerque, NM (US); Jason Wade Dugger, Albuquerque, NM (US); Zachariah Page, Austin, TX (US); Meghan Kiker, Austin, TX (US); Aaron Scarlett, Albuquerque, NM (US)

(21) Appl. No.: 18/617,817

(22) Filed: Mar. 27, 2024

Related U.S. Application Data
(60) Provisional application No. 63/455,836, filed on Mar. 30, 2023.

(51) Int. Cl. C07C 41/06

(52) U.S. Cl. CPC C07C 41/06; C07C 2603/36

(57) ABSTRACT

The invention describes a method to synthesize a divinyl- and diepoxy-substituted dibenzocyclooctanes, thereby providing a curative that can undergo a twist-boat to chair isomerization at elevated temperatures. The synthetic approach can be applied to a variety of thermosetting resins that can be crosslinked with the curative to form a polymer having a tunable coefficient of thermal expansion.

### CROSS-REFERENCE TO RELATED APPLICATION

This application claims the benefit of U.S. Provisional Application No. 63/455,836, filed Mar. 30, 2023, which is incorporated herein by reference.

### FEDERALLY SPONSORED RESEARCH AND DEVELOPMENT

This invention was made with Government support under Contract No. DE-NA0003525 awarded by the United States Department of Energy/National Nuclear Security Administration. The Government has certain rights in the invention.

### BACKGROUND OF THE INVENTION

Most solid materials experience positive thermal expansion upon heating, and the degree and rate at which this expansion occurs is referred to as the coefficient of thermal expansion (CTE). Bulk polymers typically possess large, positive CTEs in comparison to other materials. For example, a representative CTE value of an unfilled epoxy is 50-80 ppm/° C. CTE mismatch between encapsulants and underlying components often leads to cracking, delamination, or other issues. Attempts have been made to mitigate these issues with fillers such as mica or alumina. Filled epoxies (˜45 vol %) typically possess lower CTE values of 30-50 ppm/° C. However, fillers can create other issues detrimental to the material such as brittleness or stiffness.

Therefore, there is a need for thermoset and other crosslinked polymer systems that eliminate the need for fillers while achieving the CTE of a filled thermoset. In particular, there is a need for filler-less thermosets that achieve CTE tunability to near zero ppm/° C.

### SUMMARY OF THE INVENTION

The present invention is directed to a method to synthesize a divinyl- and diepoxy-substituted dibenzocyclooctanes for uses in polymers to tune coefficients of thermal expansion. The multi-step preparation of these materials starts from dibenzosuberone. Diiodination of the phenyl rings in the dibenzosuberone yields a diiodo-dibenzosuberone. The ketone in the diodinated dibenzosuberone is olefinated via a Wittig reaction and subsequent ring expansion yields a diiodo-substituted dihydrodibenzocyclooctenone. Complete reduction of the ketone yields a synthetically versatile diiodo-substituted dibenzocyclooctane. Conversion of the iodo substituents to vinyl groups to produce a divinyl-dibenzocyclooctane can be performed by palladium catalyzed cross coupling of a suitable vinyl reagent. The vinyl reagents can include those based on tin, boron, zinc, silicon or sulfoxide. The method can further comprise epoxidizing the vinyl groups of the divinyl-dibenzocyclooctane to produce a diepoxy-substituted dibenzocyclooctane. The divinyl- and diepoxy-dibenzocyclooctanes can be used to cross-link thermosetting resins.

### BRIEF DESCRIPTION OF THE DRAWINGS

The detailed description will refer to the following drawings, wherein like elements are referred to by like numbers.

FIG. 1 is a schematic illustration of thermally controlled isomerization of dibenzocyclooctane (DBCO) between twist-boat and chair conformers and how the incorporation of such contractile units into thermoset materials influences their thermal expansion and contraction behavior.

FIG. 2 shows a synthetic route to prepare divinyl- and diepoxy-DBCO and the DBCO numbering system.

FIG. 3 is a graph showing variable-temperature nuclear magnetic resonance (VT-NMR) spectra of divinyl-substituted DBCO ranging from −60° C. to 25° C.

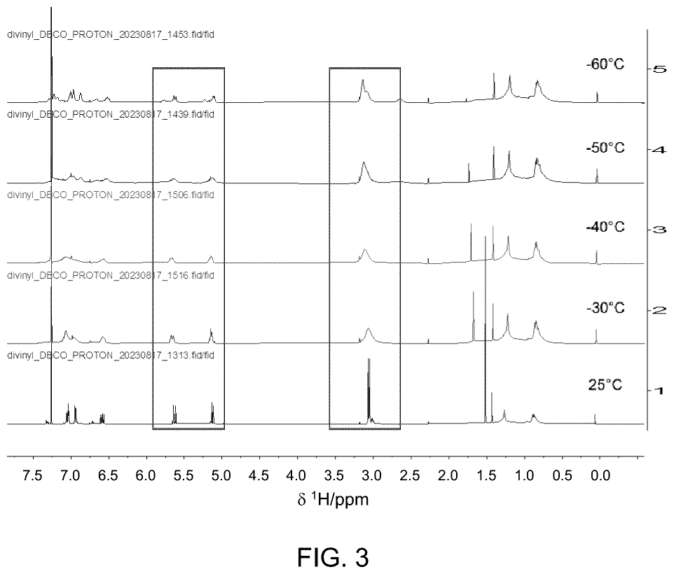

FIG. 4 shows a divinyl-substituted DBCO, siloxane monomers and catalyst used for hydrosilylation.

### DETAILED DESCRIPTION OF THE INVENTION

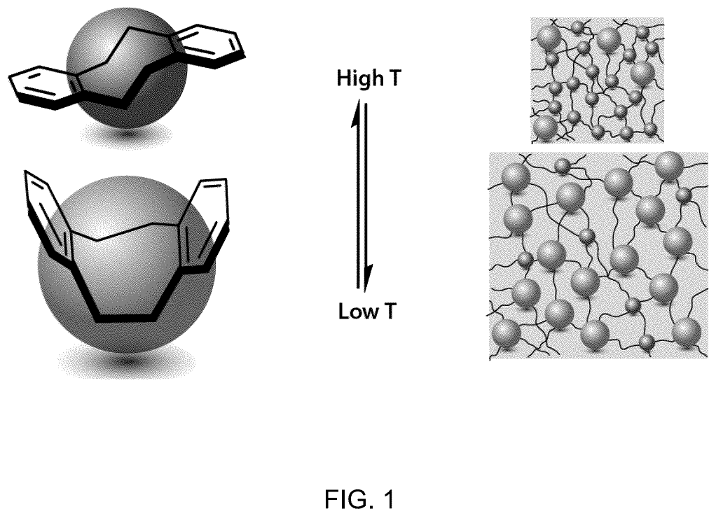

As illustrated in FIG. 1, a strategy to manipulate the CTE of thermosets involves covalent incorporation of thermally contractile units within a polymer network. See X. Shen et al., Nat. Chem. 5 (12), 1035 (2013). These “shrinking” linkages oppose thermal expansion during heating, with the net effect of reducing the CTE of the material to near or less than zero in some cases. Therefore, an approach is to synthesize materials that exhibit a tunable CTE by incorporating a molecule with high negative CTE behavior into the backbone chemistry. As shown in FIG. 1, one such contractile group is dibenzocyclooctane (DBCO), comprising a flexible cyclooctane ring connecting two rigid phenyl groups which undergoes a reversible twist-boat to chair isomerization upon heating accompanied by a decrease in molecular volume. See X. Shen et al., Nat. Chem. 5 (12), 1035 (2013); Z. Wang et al., Macromolecules 51 (4), 1377 (2018); and W. Fu et al., J. Am. Chem. Soc. 142 (39), 16651 (2020).

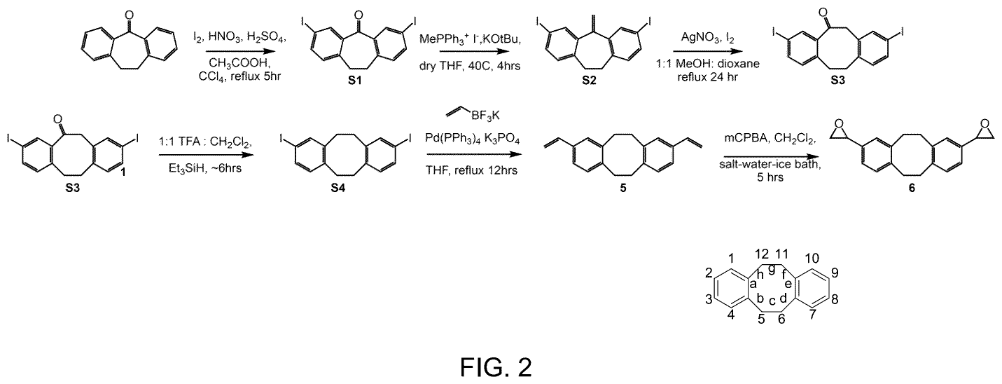

The DBCO moiety can have various reactive group substitutions on the phenyl groups. Previous density-functional theory (DFT) calculations on model DBCO compounds revealed a temperature-dependent isomerization equilibrium that was most significant for the cis-diamino-DBCO (DADBCO) regioisomer. The cis terminology refers to the orientation of the amino groups with respect to one another in the DBCO ring system and would be substituted at the 2 and 9 positions according to the numbering scheme in FIG. 2. Consistent with the DFT data, an epoxy cured with cis-DADBCO possessed a low CTE value of 20 ppm/° C. below its T_{g} and contracted massively above T_{g}, amounting to a net contraction of the material over the tested temperature range that was highly reversible. See U.S. Pat. No. 11,739,092, which is incorporated herein by reference. Therefore, the cis-disubstituted-DBCO molecule is preferred. The DBCO molecule can be substituted with a variety of reactive groups, including amine, 2-hydroxyethyl, vinyl, epoxide, etc., thereby providing a curative that can undergo a twist-boat to chair isomerization at elevated temperatures. The synthetic approach can be applied to a variety of thermosetting resins that can be crosslinked with the curative to form a thermoset, including but not limited to epoxy resins, acrylates, methacrylates, unsaturated polyesters, vinyl esters, urethanes, silicones, and siloxanes, and other crosslinked polymer systems to provide a tunable coefficient of thermal expansion.

An embodiment of the invention is directed to the regioselective synthesis of a DBCO framework that results in 2,9-functionalized (cis) derivatives. The invention is specifically directed to divinyl and diepoxy derivatives synthesized using a regioselective method. A generalized method to synthesize the divinyl- and diepoxy-DBCOs is shown in FIG. 2. The first three steps in the synthesis to make compounds S1-S3 were followed as reported by Kardelis et al., however, some modifications were made to optimize reaction time and work up. See V. Kardelis et al., Angew. Chem. Int. Ed. 55 (3), 945 (2016), which is incorporated herein by reference. The first step, diiodination of dibenzosuberone 1, was performed with no modifications. Next, the ketone of S1 was converted to an alkene via a Wittig reaction to produce olefinated di(iodobenzo)cycloheptane S2 so that it could undergo a subsequent ring expansion. Conditions for the Wittig reaction were followed as reported in literature, with the only optimization being reduced reaction time (yield ˜70%). Ring expansion conditions were followed as reported in literature to produce di(iodobenzo)cyclooctanone S3. Purification for this step was modified to a precipitation in methanol over flash column chromatography without a loss in yield (˜66%).

Reduction of the ketone in ring expanded compound S3 was accomplished using triethylsilane (TES)/trifluoracetic acid (TFA). See P. Li et al., Organic Lett. 16 (1), 182 (2014), which is incorporated herein by reference. The reduction by TES/TFA resulted in the high yield (80%) of the desired compound, di(iodobenzo)cyclooctane S4. Compared to Wolff Kishner or LiAlH₄ reduction chemistries, the reduction as described is technically less demanding because of milder conditions, shorter reaction times, and the use of less harmful reagents.

The Suzuki-Miyaura cross-coupling reaction was used to install vinyl groups onto the DBCO framework as a platform for several potential transformations. Palladium catalyzed vinylation of diiodinated DBCO S4 with potassium vinyltrifluoroborate gave divinyl-DBCO 5. Conditions for the reaction were followed as generally reported in literature, using compound S4 and an excess of potassium vinyltrifluoroborate as the coupling reagents along with a low equivalent of Pd(PPh₃)₄ catalyst in a biphasic solvent system of tetrahydrofuran (THF) and aqueous tribasic potassium phosphate (2 M). No phase transfer catalysts are required. The reaction was successful in high yield (˜80%) with a simple work up and purification via a silica plug to isolate divinyl DBCO 5. Divinyl-DBCO 5 can be epoxidized using m-chloroperoxybenzoic acid (mCPBA) to give diepoxy-DBCO 6. Synthesis of the specific intermediates is described below.

Synthesis of 3,7-diiodo-5H-dibenzo[a,d]cycloheptan-5-one S1. The synthesis was accomplished in an analogous fashion to that previously reported by Kardelis et al. See V. Kardelis et al., Angew. Chem. Int. Ed. 55 (3), 945 (2016). Solvent was removed by rotary evaporation to obtain a red-brown solid. The product was purified by dissolving in a minimal amount of 1:1 CHCl₃:Hx and filtered. The precipitate was collected and recrystallized in EtOH to obtain an off white crystalline solid (28%). HRMS (ESI): exact mass calculated for C₁₅H₁₀I₂O 459.8821, found 459.8814.

Synthesis of 3,7-diiodo-5-methylene-5H-dibenzo[a,d]cycloheptane S2. The synthesis was accomplished in an analogous fashion to that previously reported by Kardelis et al. See V. Kardelis et al., Angew. Chem. Int. Ed. 55 (3), 945 (2016). The product was purified by flash chromatography (4:96 DCM:Hx) to obtain a white powder (70%). HRMS (ESI): exact mass calculated for C₁₆H₁₂I₂ 457.9028, found 457.9031.

Synthesis of 3,8-diiodo-dibenzo[a, e]cyclooctan-5 (6H)-one S3. The synthesis was accomplished in an analogous fashion to that previously reported by Kardelis et al. See V. Kardelis et al., Angew. Chem. Int. Ed. 55 (3), 945 (2016). After removal of Agl by filtration, the reaction solution was concentrated by rotary evaporation and was purified by precipitation in MeOH. The precipitate was filtered and washed with MeOH to obtain a white powder (60%). HRMS (ESI): exact mass calculated for C₁₆H₁₂I₂O 473.8978, found 473.8986.

Synthesis of 2,9-diiodo-dibenzo[a, e]cyclooctane S4. To a round bottom flask was added S3 (6.32 g, 13.3 mmol), DCM (47 mL), and trifluoroacetic acid (TFA) (47 mL). Triethylsilane (TES) (21.3 mL, 133 mmol) was slowly added to the solution and let stir at room temperature for 5 h. The reaction was monitored by TLC (R_{f} of 0.8 in 1:4 DCM:Hx). Upon completion, the reaction was added to a large amount of DI water and neutralized with sodium bicarbonate (checked with pH strip). The solution was then extracted with DCM (3×50 mL). The organic phase was washed with water (3×50 mL) and brine (50 mL) and dried over MgSO₄. The solution was filtered and concentrated by rotary evaporation Recrystallization from acetonitrile yielded a white powder S4 (4.9 g, 80%). HRMS (ESI): exact mass calculated for C₂₀H₂₀ 459.9185, found 459.9182.

Synthesis of 2,9-divinyl-dibenzo[a, e]cyclooctane 5. To a two-neck round bottom flask was added THF (12.00 mL) and 2 M tripotassium phosphate (12.00 mL, aqueous). The reaction was degassed with argon for 5 min before adding compound S4 (1.07 g, 2.33 mmol), potassium vinyltrifluoroborate (1.25 g, 9.30 mmol), and Pd(PPh₃)₄ (134 mg, 116 μmol). The reaction was degassed with argon for another 5 min, then heated to reflux for 12 h with vigorous stirring. Upon completion, the reaction was concentrated by rotary evaporation to remove THF and extracted with DCM (3×50 mL). The organic layer was washed with H₂O (3×50 mL), brine (50 mL), dried over MgSO₄, and filtered. The product was purified via silica plug using 1:99 DCM:Hx to load and rinsed with Hx. Solvent was removed by rotary evaporation to obtain a white powder (606 mg, 80%). HRMS (ESI): exact mass calculated for C₂₀H₂₀ 260.1565, found 260.1559.

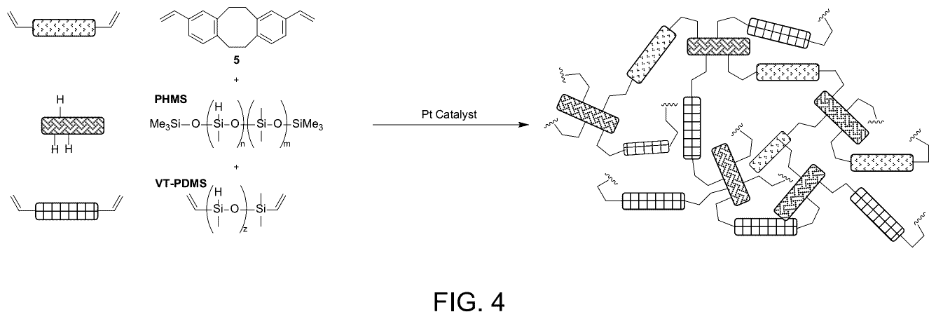

Variable-temperature nuclear magnetic resonance (VT-NMR) was used to investigate the conformation change of divinyl-DBCO from boat-to-chair based on findings by Fu et al. See W. Fu et al., J. Am. Chem. Soc. 142 (39), 16651 (2020). FIG. 4 is a graph showing VT-NMR of divinyl-DBCO ranging from −60° C. to 25° C. This graph shows a low energy conformation change occurring around −60° C.

The divinyl-DBCO regioisomer was also investigated for CTE behavior via hydrosilylation with polymethylhydrosiloxanes (PHMS and VT-PDMS), shown in FIG. 4. A span of polydimethylsiloxane samples with varying amounts of divinyl-DBCO (0-50%) and 5% crosslinked with tetramethyl-tetravinyl-cyclotetrasiloxane were prepared using commercially available monomers to emulate commercially available silicone resins. Monomers were mixed with Karstedt's catalyst (if needed, toluene may be added to aid in dissolution of divinyl DBCO), poured into a silicone mold, and cured at 100° C. in an oven for 1 h minimum. To remove any toluene left in the sample, the samples were further dried at 100° C. in under vacuum. The samples were characterized by dilatometry to evaluate CTE behavior in the temperature range of −90 to 0° C. The results are shown in Table 1.

TABLE 1
Coefficient of thermal expansion for
divinyl-DBCO polydimethylsiloxanes.

| DV-DBCO content (%) | CTE (10⁻⁶/K) |
|---|---|
| 0 | 232 |
| 20 | 188 |
| 50 | 120 |

The present invention has been described as methods to synthesize divinyl- and diepoxy-substituted DBCOs for use in polymers with tunable coefficients of thermal expansion. It will be understood that the above description is merely illustrative of the applications of the principles of the present invention, the scope of which is to be determined by the claims viewed in light of the specification. Other variants and modifications of the invention will be apparent to those of skill in the art.

1. A method to synthesize a substituted dibenzocyclooctane, comprising:
providing a dibenzosuberone;
diiodinating the phenyl rings of the dibenzosuberone to produce a diiodo-dibenzosuberone;
olefinating the ketone of the diiodo-substituted dibenzosuberone to produce a diiodo-methylene-benzocycloheptane;
ring expanding the diiodo-methylene-benzocycloheptane to produce a diiodo-substituted dibenzocyclooctanone;
reducing the ketone of the diiodo-substituted dibenzocyclooctanone using a triethylsilane/trifluoroacetic acid mixture to produce a diiodo-substituted dibenzocyclooctane;
converting the iodo substituents of the diiodo-substituted dibenzocyclooctane to vinyl groups to produce a divinyl-substituted dibenzocyclooctane.

2. The method of claim 1, further comprising epoxidizing the vinyl groups of the divinyl-substituted dibenzocyclooctane to produce a diepoxy-substituted dibenzocyclooctane.

3. The method of claim 1, wherein the divinyl-substituted dibenzocyclooctane comprises a dibenzocyclooctane substituted with vinyl groups at the 2 and 9 positions.

4. The method of claim 1, further comprising crosslinking a thermosetting resin with the divinyl-substituted dibenzocyclooctane to form a thermoset.

5. The method of claim 4, wherein the thermosetting resin comprises an epoxy, acrylate, methacrylate, unsaturated polyester, vinyl ester, urethane, silicone, or siloxane.
